(19) United States
(12) Patent Application Publication
(10) Pub. No.: US 2009/0005869 A1
(43) Pub. Date: Jan. 1, 2009

(54) Device which Attaches into a Joint and Carries a Payload of Controlled Release Drugs and Related Method thereof

(75) Inventors: Cato T. Laurencin, Earlysville, VA (US); S. Raymond Golish, Charlottesville, VA (US); Lakshmi Sreedharan Nair, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 11/957,088

(22) Filed: Dec. 14, 2007

Related U.S. Application Data
(60) Provisional application No. 60/875,020, filed on Dec. 15, 2006.

(51) Int. Cl. A61F 2/02; A61B 17/04; A61M 31/00

(52) U.S. Cl. 623/11.11; 606/232; 604/57

(57) ABSTRACT

A device that is secured inside a human or animal joint. The device may include, but not limited thereto, the following components: 1) eyelets for suture; 2) endcaps; 3) a longitudinal core; 4) optional additional longitudinal members; and 5) a space for carrying a payload of controlled release drug. Numerous preparations of controlled release drugs have been proposed. This device acts as a platform for said preparations and drugs. It is distinguished by, among other things, its geometry and structural elements which are optimized for: 1) surgical passage into a joint; 2) stable attachment inside of a joint; and 3) secure carriage of the payload device for controlled release of drug.

### RELATED APPLICATIONS

The present patent application claims priority from U.S. Provisional Application Ser. No. 60/875,020, filed Dec. 15, 2006, entitled “Device which Attaches into a Joint and Carries a Payload of Controlled Release Drugs and Related Method thereof,” the entire disclosure of which is hereby incorporated by reference herein in its entirety.

### FIELD OF THE INVENTION

The invention relates to the field of devices for surgical or procedural implantation into a joint. The device secures itself into the joint to carry a payload of controlled release preparations of drugs.

### BACKGROUND OF THE INVENTION

The administration of drugs directly to the joints of humans and animals is an important therapy for diseases such as osteoarthritis and rheumatoid arthritis. Local anaesthetics, corticosteroids, and hyaluronic acid preparations are widely administered to the joints of humans and animals, including the knee, shoulder, hip, and other joints. Intra-articular administration (directly to the joint) can increase the effect and reduce the side effects over other routes of administration.

Controlled release preparations of drugs are proliferating. However, most drugs remain in a joint for a short period of time, from hours to weeks. Consequently, their effects range from days to months, considerably shorter than is desired. Various preparations involving polymers, macromolecules, microdevices, nanodevices and assemblies of such components have been engineered.

A goal of this work is to store a dose of drug that is slowly released over an extended period of time, from days to years.

Some controlled release drug preparations can be administered by injection of microscopic particles or gels directly into the joint. However, a separate class of controlled release drug preparations are fabricated into macroscopic pellets, beads, cylinders, wafers, or other devices. These solid preparations have several advantages, but must be implanted surgically.

Solid preparations of controlled release drugs cannot be left unsecured in a joint. A joint is a dynamic mechanical environment experiencing the forces of locomotion.

A device and related method is needed that provides a stable platform to carry the drug delivery device itself. Such a carrier device may have, for example, a geometry and structural elements that are optimized for 1) easy surgical passage into a joint; 2) stable attachment inside of a joint; and 3) secure carriage of the payload drug.

Several groups have described devices for carrying a payload of controlled release drug in a joint. Watson, et al. allegedly describe a device involving a bone screw with a chamber for receiving a drug payload (US patent application 2005/0031665); this is related to an earlier application from the same group (2002/0169162). Watson, et al. allegedly describe screws, tacks and staples with a small chamber. These devices are to be inserted into bone and carry the drug in the chamber. Hotchkiss, et al. allegedly describe a device involving removal of bone or cartilage to create a receptacle space for implantation of a device that is flush with the surface (US patent application publication 2005/0152949).

Other groups have described devices for carrying a payload of controlled release drug in other body spaces. Jacobsen, et al (U.S. Pat. No. 6,530,934) allegedly describe a device for delivery of drug to the intravascular space. The device is a string of beads of controlled release drug. Weiner, et al. allegedly describe a tack for drug delivery to the intraocular space (U.S. Pat. No. 5,466,233). Several groups have disclosed drug pumps and other related devices (e.g., U.S. Pat. No. 6,901,287). Watson, et al. allegedly describe a device with a chamber and a suture tag for intraocular implantation that hold a payload in an impermeable chamber that elutes through a hole (US patent application publication 2005/0158365). Levy et al. allegedly describe a related device (US patent application publication 2005/0136095). De Carvalho et al. also allegedly describe a chambered intraocular device (US patent application publication 2005/0113806).

Various groups have disclosed devices which are controlled release preparations of drugs involving polymers, macromolecules, microdevices, nanodevices and assemblies of such components; these compositions may be fabricated into macroscopic pellets, beads, cylinders, wafers, or other payload devices. They may also be fabricated into functional devices, such as tacks, screws, eyelets, and sutures, alone or as a part of a blend of other polymers.

For example, Tuan et al. allegedly describe fracture fixation devices fabricated from PLAGA polymer melt casted with fluoroquinolone-type antibiotics (U.S. Pat. No. 5,281,419). Tormala et al. allegedly describe fracture fixation devices of polymer, antibiotic, and glass fibers (US patent application 2004/0009228). Grisoni et al. allegedly describe a bioabsorbable string of calcium sulfate beads loaded with drug (U.S. Pat. No. 5,756,127). Elkhoury et al. allegedly describe a suture device composed of polymer that elutes opiod-type analgesic drugs (U.S. Pat. No. 5,919,473). Dave et al. allegedly describe a layered device that both elutes drug and controls pH (US patent application 20050267565). Carr et al. allegedly describe rods of polymers and a blend of analgesic drugs (U.S. Pat. No. 6,913,760).

Numerous groups have allegedly disclosed devices which are controlled release preparations of drugs involving polymers, macromolecules, microdevices, nanodevices and assemblies of such components; these are disclosures of the microscopic compositions irrespective of their fabrication into macroscopic devices or carrier devices for such preparations (e.g. U.S. Pat. Nos. 5,922,340; 6,921,541; 6,699,908; 7,052,719; 6,534,081).

Many groups have allegedly described devices related to the general field of joint surgery or to the field of arthroscopic joint surgery in which joint surgery is accomplished by means of devices and instruments inserted through small portals. Some of these device related to securing tissues, tying knots, employing bone anchors, or otherwise manipulating the joint with small devices and instruments. These are not for the sole purpose of securing controlled release drugs (e.g. U.S. Pat. Nos. 7,037,324; 7,001,411; 6,932,834; 6,923,823; 6,749,620; 6,592,609; 6,626,916).

### SUMMARY OF THE INVENTION

Various aspects of embodiments of the present invention may comprise a device and method that are provided which are useful for the carriage of controlled release drugs to the human knee joint or shoulder joint. It is recognized that the device and method can be used in other applications to any human or animal diarthrodial joint.

A device (and related method) that is secured inside a human or animal joint. The device may include, but not limited thereto, the following components: 1) eyelets for suture; 2) endcaps; 3) a longitudinal core; 4) optional additional longitudinal members; and 5) a space for carrying a payload of controlled release drug. Numerous preparations of controlled release drugs have been proposed. This device acts as a platform for said preparations and drugs. It is distinguished by, among other things, its geometry and structural elements which are optimized for, but not limited thereto, the following: 1) surgical passage into a joint; 2) stable attachment inside of a joint; and 3) secure carriage of the payload device for controlled release of drug.

In one aspect of the invention, there is a device provided that contains multiple structural members. The members are composed of a solid material with advantageous mechanical properties. An overall purpose of the device is to carry a second, separate device which delivers a payload of controlled release drugs. Such a device is advantageous as a means of, but not limited thereto, stably securing the payload, and as a means of mechanically protecting the payload from the forces of joint motion.

In one general aspect of the invention, the carrier device has mechanical properties superior to the payload device viz. higher stiffness, decreased brittleness, higher yield strength, higher ultimate strength, higher strength to failure, or other properties. In the case that the disclosed device is constructed of a polymer, the carrier device may have polymeric properties superior to the payload device viz. surface erosion, bulk erosion, crystallinity, degradation constants, diffusion constants, co-polymer ratios, polymer blend ratios, or other properties. In one general aspect of the invention, the device may be constructed of polymers, ceramics, or metals, or compositions of these materials. Preferably, the device is constructed of bioerodible or biodegradable polymers which are biocompatible.

Various groups have disclosed payload devices for controlled release of drugs. The present invention carrier device (and related method) accommodates a variety of payload devices which may be disclosed elsewhere in the academic and patent literature. An example of a payload device is a pellet of drug, filler, and binder compressed into a payload device by a pharmaceutical tablet press. The payload technology is capable of producing controlled release devices when used with a suitable coating. More complex payload devices can be fabricated from blends of polymers, drugs, and other carriers, such as those shown in, for example, but not limited thereto, U.S. Pat. No. 5,919,473 to Elkhoury and U.S. Pat. No. 6,913,760 to Carr et al.

In one general aspect of the invention, there is a method for securing the device to suture, preferably the existence of suture eyelets at two opposing positions on the device. The suture eyelets hold suture, and the suture is used to attach to bony and soft tissue structure within the joint to secure the device. It should be appreciated that the suture may be secured in the joint by numerous method, including suturing joint capsule, open knot tying, arthroscopic knot tying, bone anchors which are distinct from the invention, or the like. It will be appreciated that other methods of securing suture to the device are contemplated within the scope of the invention.

In one general aspect of an embodiment of the invention, the geometries contemplated are optimized for insertion through small incisions. In an exemplary embodiment, the structural members are comprised of the following: 1) core members which are one or more rods oriented longitudinally 2) endcap members at both ends of the core members for structural support of the core and protection of the payload device; 3) suture eyelet members continuous with the endcap members which affix suture to the device; 4) a void space for carriage of the payload drug. It will be appreciated that other embodiments may include core members oriented centrally or peripherally with respect to the endcap members.

In an exemplary embodiment, the device resides in the axillary recess of the shoulder joint, the lateral or medial gutter of the knee joint, or the suprapatellar pouch of the knee joint. It will be appreciated that other position in other joints are contemplated.

An aspect of an embodiment of the device is that it has been configured to withstand the dynamic mechanical environment experienced during the typical forces of locomotion. An aspect of an embodiment of the device is that it has allows a dose of drug to be slowly released on the time scale of weeks to years, as opposed to current technology that works on the time scale of hours to days. The device can be easily, securely, and conveniently implanted directly into any diarthrodial joint. Once implanted, the apparatus acts to protect the payload from the dynamic environment within the joint to allow for extended, controlled drug delivery.

An aspect of an embodiment of the present invention provides a carrier device for delivery of drugs to a diarthrodial joint of a human or animal subject. The carrier device comprising multiple structural members, whereby the multiple structural members are adapted to: secure the carrier device to the tissues of the joint space to stabilize the device within the joint; and for delivering a payload of a separate device.

An aspect of an embodiment of the present invention provides a method for delivery of drugs to a diarthrodial joint of a human or animal subject using a carrier device. The method comprising: securing the carrier device to the tissues of the joint space to stabilize the device within the joint, and delivering a payload of a separate device.

These and other objects, along with advantages and features of the invention disclosed herein, will be made more apparent from the description, drawings and claims that follow.

### BRIEF DESCRIPTION OF DRAWINGS

The accompanying drawings, which are incorporated into and form a part of the instant specification, illustrate several aspects and embodiments of the present invention and, together with the description herein, and serve to explain the principles of the invention. The drawings are provided only for the purpose of illustrating select embodiments of the invention and are not to be construed as limiting the invention.

FIG. 1(A) provides an end elevation view of an embodiment of the present invention device.

FIG. 1(B) provides a front elevation view of an embodiment of the present invention device.

FIG. 1(C) provides a perspective view of an embodiment of the present invention device.

FIG. 1(D) provides a top plan view of an embodiment of the present invention device.

FIG. 2 provides a perspective view of an embodiment of the present invention device of FIG. 1 illustrated by shading to indicate stress and strain of the embodiment under cantilever bending.

FIG. 3(A) provides an end elevation view of an embodiment of the present invention device.

FIG. 3(B) provides a front elevation view of an embodiment of the present invention device.

FIG. 3(C) provides a perspective view of an embodiment of the present invention device.

FIG. 3(D) provides a top plan view of an embodiment of the present invention device.

FIG. 4 provides a perspective view of an embodiment of the present invention device of FIG. 3 illustrated by shading to indicate stress and strain of the embodiment under cantilever bending.

FIG. 5 provides a frontal elevation view demonstrating a longitudinal core member, endcap members, and suture attachments.

FIG. 6 provides a frontal elevation view demonstrating a central longitudinal core member and eccentric, peripheral longitudinal members.

FIG. 7 provides a frontal elevation view demonstrating a peripheral longitudinal member consisting of a cylindrical shell which enables absence of a central longitudinal member.

FIG. 8(A) provides a frontal elevation view of suture integrated with a structural member.

FIG. 8(B) provides a frontal elevation view of suture integrated with a structural member.

FIG. 9 provides a frontal elevation of suture attached through suture eyelet members to a longitudinal core member.

FIG. 10 provides a photographic of a fabricated prototype device from FIG. 1.

### DETAILED DESCRIPTION OF THE INVENTION

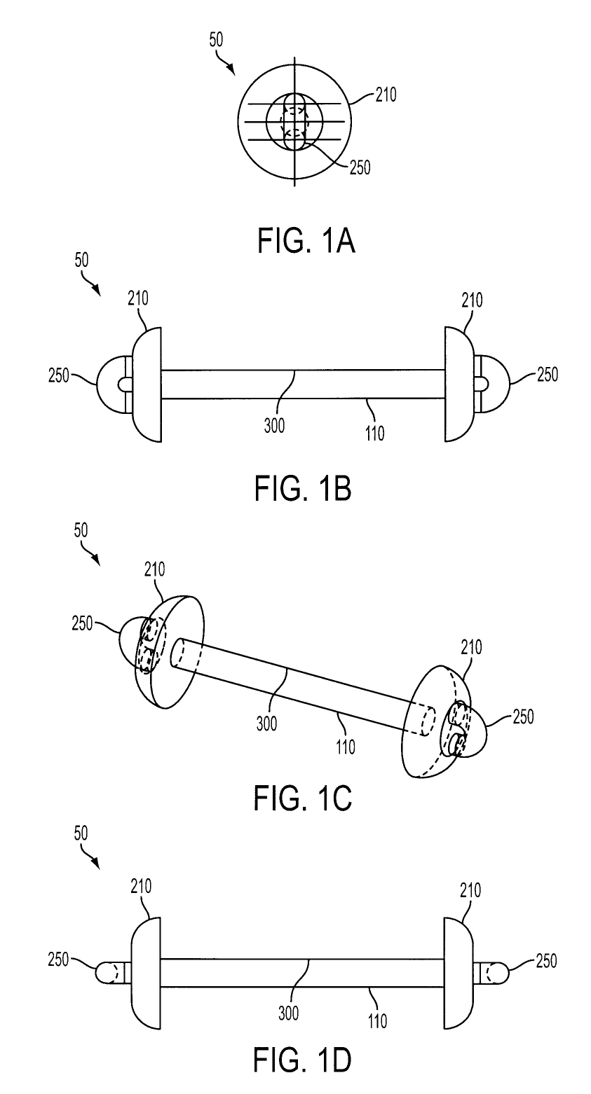

In an exemplary embodiment, FIGS. 1(A)-1(D) demonstrates an optimized device 50 containing a longitudinal core member 110, such as a cylindrical core. The core member 110 may have a diameter of about 2 mm and of about length of 20 mm. The core member 110 supports two disc-shaped endcaps 210. The endcaps may have an outer diameter of about 4 mm, an inner diameter of about 2 mm, an outer fillet with a radius of about 2 mm, and a height of about 2 mm. The endcap members each support a suture eyelet member 250. The eyelet member includes a half-torus with an inner diameter of 1 mm and an outer diameter of 3 mm. The half-torus is supported atop two cylindrical posts of diameter about 2.5 mm and height about 0.5 mm which affix to the endcap members to extend the eyelet inner diameter in the longitudinal direction. The total eyelet inner diameter in the longitudinal direction may be about 1 mm. The total payload of the device may be approximately 1 cubic centimeter (94.3 cubic millimeters). Item 300 represents a payload area for a payload device (not shown) having a drug, payload material/structure or the like. The payload area 300 is a cavity or area that represents the absence of any other physical design elements. It is the space or area into which, for example, a pellet, payload, or engineered device of controlled delivery drug may be inserted. The payload device (not shown) may be inserted into and attached to the disclosed carrier device by any combination of sutures, tabs, tangs, adhesive, or other structural design elements as desired or required.

An example of a payload device is a pellet of drug, filler, and binder compressed into a payload device by a pharmaceutical tablet press, for example. The payload technology is capable of producing controlled release devices when used with a suitable coating. More complex payload devices can be fabricated from blends of polymers, drugs, and other carriers, such as those shown in, for example, U.S. Pat. No. 5,919,473 to Elkhoury and U.S. Pat. No. 6,913,760 to Carr et al. It should be appreciated that a variety of payload technology (devices, materials, drugs, structures, etc.) may be implemented with the present invention carrier devices as desired or required.

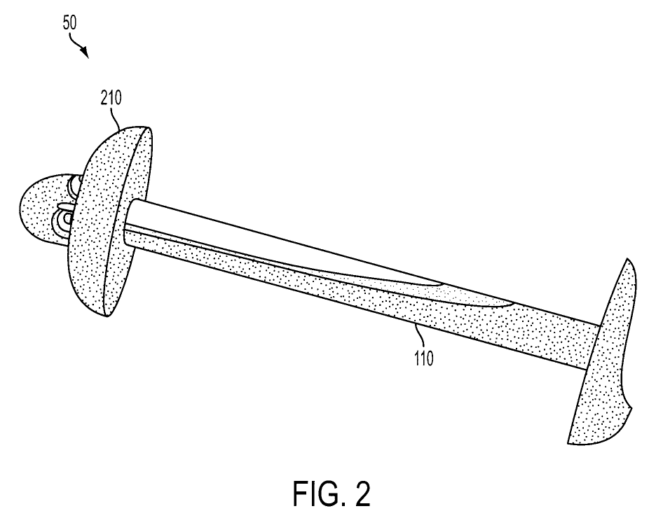

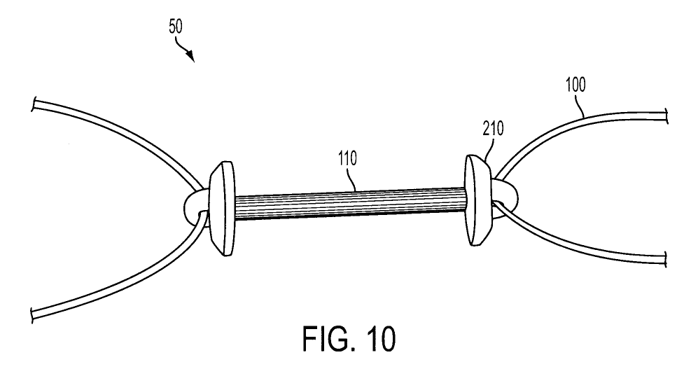

In an exemplary embodiment, FIG. 2 demonstrates the stress distribution and deformation of the device from FIG. 1 under a bending moment applied at the suture eyelet with a constraint at the opposite suture eyelet. Stress is highest at the interface between eyelet and endcap, and along the distal shaft of the longitudinal core member. In an preferred embodiment, FIG. 10 is a photographic of a fabricated prototype device as shown in FIG. 1 with about USP #2 diameter suture running through the eyelets (about 500 micrometer polyester suture). The various shades of grey denote specific stress values in a computer simulation of the mechanics of an embodiment the present invention device. The grey shading generally provides the relative relationships of the stress values.

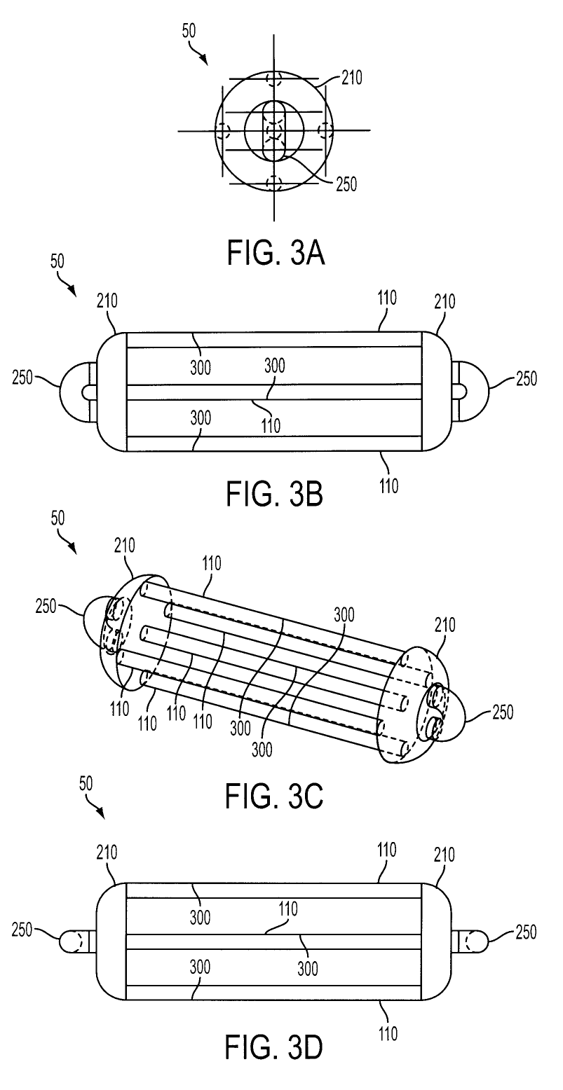

In another embodiment, FIG. 3 demonstrates an optimized device of similar geometry to the device 50 in FIG. 1. However, FIG. 3 demonstrates five longitudinal core members 110, such as a cylindrical core. The core members may have a diameter of about 1 mm and a length of about 20 mm. The core member support two disc-shaped endcaps 210, each supporting a suture eyelet 250. The endcaps 210 and eyelets 250 may be of the geometries and dimensions described in FIG. 1. Item 300 represents a payload area for a payload device (not shown) having a drug, payload material/structure or the like. The payload area 300 is a cavity or area that represents the absence of any other physical design elements. It is the space or area into which, for example, a pellet, payload, or engineered device of controlled delivery drug may be inserted. The payload device (not shown) may be inserted into and attached to the disclosed carrier device by any combination of sutures, tabs, tangs, adhesive, or other structural design elements as desired or required.

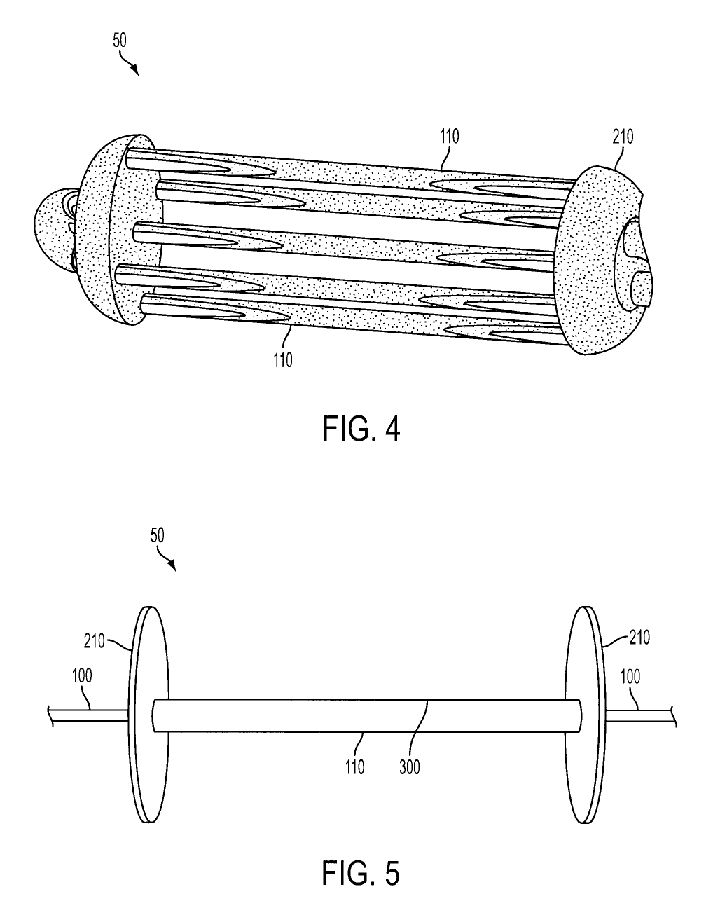

With respect to FIG. 3, FIG. 4 demonstrates the stress distribution and deformation of the device from FIG. 3 under a bending moment applied at the suture eyelet with a constraint at the opposite suture eyelet. Stress is highest at the interface between eyelet and endcap, and along the distal shaft of the longitudinal core members. The various shades of grey denote specific stress values in a computer simulation of the mechanics of an embodiment the present invention device. The grey shading generally provides the relative relationships of the stress values.

In one general aspect of the invention of the present invention device 50, FIG. 5 demonstrates structural members. There are one or more longitudinal core members 110, such as a cylindrical core. Endcap members affix to each end of the core member 210. Suture 100 is attached to the device 50 either by suture eyelets affixed to each endcap member, or suture may be integrated with solid members as described below. Item 300 represents a payload area for drug. Item 300 represents a payload area for a payload device (not shown) having a drug, payload material/structure or the like. It is the space or area into which, for example, a pellet, payload, or engineered device of controlled delivery drug may be inserted. The payload device (not shown) may be inserted into and attached to the disclosed carrier device by any combination of sutures, tabs, tangs, adhesive, or other structural design elements as desired or required.

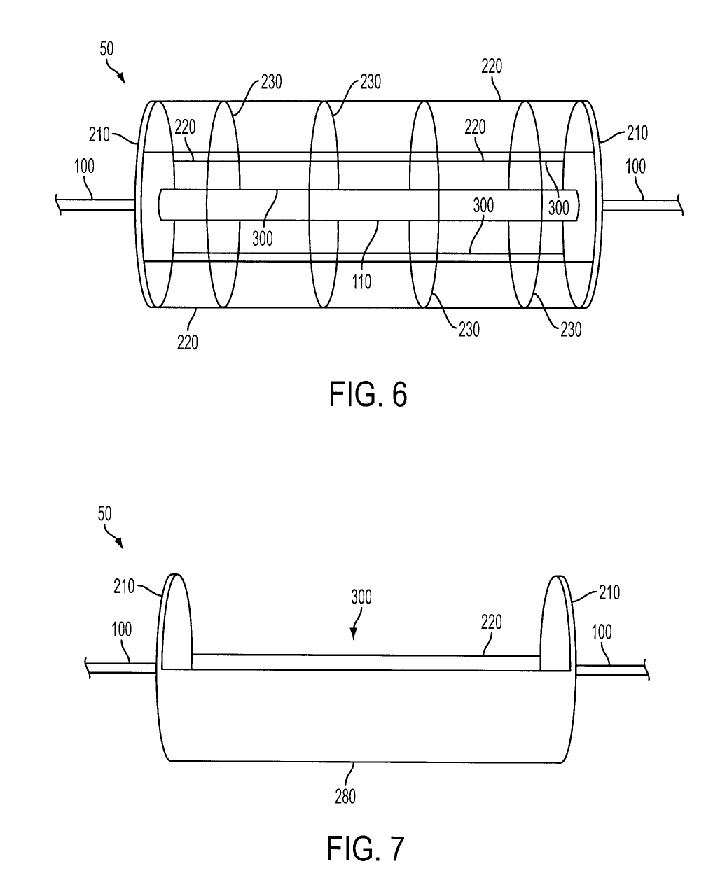

In one general aspect of an embodiment of the present invention device 50, FIG. 6 demonstrates that one or more longitudinal core members may be placed centrally, referenced as 110 or eccentrically and peripherally, as referenced as 220, with respect to the endcap members 210. Multiple longitudinal core members may be integrated with circumferential hoops 230. In one embodiment, longitudinal members and hoop members are numerous and of small caliber, creating a screen or mesh circumferentially about the endcap. Item 300 is the payload area for drug. The payload area 300 is a cavity or area that represents the absence of any other physical design elements. It is the space or area into which, for example, a pellet, payload, or engineered device of controlled delivery drug may be inserted. The payload device (not shown) may be inserted into and attached to the disclosed carrier device by any combination of sutures, tabs, tangs, adhesive, or other structural design elements as desired or required.

In another embodiment of the invention of the present invention device 50, FIG. 7 demonstrates that members may be peripherally placed in the form of a cylindrical shell 280, affixed to the endcaps 210. In one general aspect of the invention, the placement of peripheral or eccentric longitudinal core members 280, may not necessitate a central longitudinal core member (not shown). Item 300 is the payload area for drug and item 100 is attached suture. The payload area 300 is a cavity or area that represents the absence of any other physical design elements. It is the space or area into which, for example, a pellet, payload, or engineered device of controlled delivery drug may be inserted. The payload device (not shown) may be inserted into and attached to the disclosed carrier device by any combination of sutures, tabs, tangs, adhesive, or other structural design elements as desired or required.

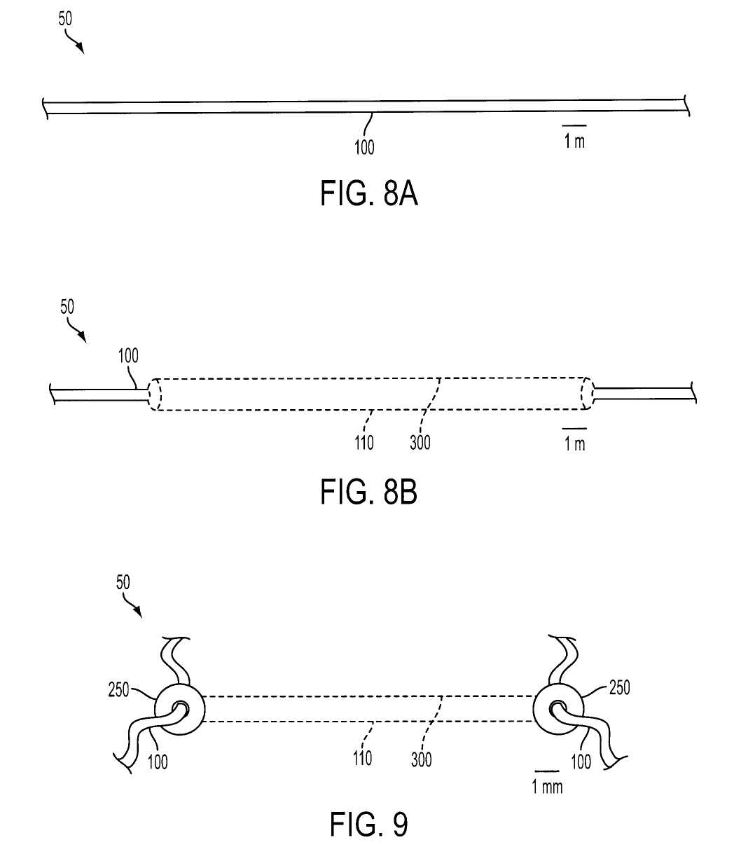

In one general aspect of the invention of the present invention device 50, FIG. 8 demonstrates that suture 100 is attached to the device (FIG. 8(A)). In one embodiment, suture 100 may be integrated into one or more structural elements by means of casting/molding or other manufacturing methods. In another embodiment, suture item 100 is continually integrated internal to a longitudinal core element 110 (FIG. 8(B)), such as a cylindrical core. In this embodiment, endcap elements and suture eyelet elements are eliminated. In another embodiment, suture may be integrated into endcap elements (not shown) supporting one or more longitudinal core elements 110 placed centrally or eccentrically and peripherally.

In another embodiment of the present invention device 50, FIG. 9 demonstrates the passage of suture 100) through suture eyelet members250. In one embodiment, the eyelet members 250 are affixed to a longitudinal core member 110 (as shown), such as a cylindrical core. In this embodiment, endcap elements are eliminated. In other embodiments, suture eyelets affix to endcap elements (not shown).

In one general aspect of an embodiment the present invention, the device contains structural elements. Structural elements include one or more longitudinal core elements, one or more endcap elements, and one or more suture eyelet elements. One or more groups of elements is present, but not all of the elements are present in some embodiments. In one general aspect of an embodiment of the present invention, the device will be attached to suture. The suture may be passed though said suture eyelets or integrated into one or more structural elements of the device without the use of eyelets.

In one general aspect of an embodiment of the present invention, longitudinal core members may be of several geometries including a cylinder, prism, cylindrical shell, section of a pyramidal solid, or partial arc of these geometries. Longitudinal core members may be circumferentially integrated by means of hoops or a screen or mesh for protection of payload drug. Endcaps members may be of several geometries, including a concave-concave disc, a concave-convex disc, a convex-convex disc, a cylinder, a prism, a rotated base, an extruded base, or a fillet-modified version of the above geometries. Suture eyelet members may be of several geometries including a torus, an arc of a torus, connected prisms, connected cylinders, a rotated base, an extruded base, an embossed base or a combination of these elements.

In one general aspect of an embodiment of the present invention, the carrier device has mechanical properties superior to the payload device, either due to the geometry of the carrier device or the material properties of the carrier device, viz. higher stiffness, decreased brittleness, higher yield strength, higher ultimate strength, higher strength to failure, or other properties. In the case that the disclosed device is constructed of a polymer, the carrier device may have polymeric properties superior to the payload device viz. surface erosion, bulk erosion, crystallinity, degradation constants, diffusion constants, co-polymer ratios, polymer blend ratios, or other properties. In one general aspect of an embodiment of the present invention, the device may be constructed of polymers, ceramics, or metals, or compositions of these materials. Preferably, the device may be constructed of bioerodible or biodegradable polymers which are biocompatible.

In one embodiment of an embodiment of the present device, the structural members are constructed of a bioerodible or biodegradable polymers which are biocompatible but also contain controlled release drugs. The polymers and drugs may be distinct from or identical to the payload drug. The payload drug may be structurally separate or entirely integrated with the payload device.

In an embodiment, the device resides in the axillary recess of the shoulder joint, the lateral or medial gutter of the knee joint, or the suprapatellar pouch of the knee joint. It will be appreciated that other position in other joints are contemplated.

It should be appreciated that various sizes, dimensions, contours, rigidity, shapes, flexibility and materials may be varied and utilized as desired or required.

It should be appreciated that any of the components, elements, sizes, characteristics, integrations, separateness, disposability, detachability, integration, and functions associated with any of the embodiments (or its components or sub-components) explicitly taught or suggested or inferred may be interchanged, added, removed, augmented, resized, contoured, or replaced with any of the components, elements, sizes and functions associated with other respective embodiments herein. Such components, elements, sizes, characteristics, integrations, separateness, disposability, detachability, integration, and functions may include, but not limited thereto, the following reference numbers: 50, 100, 110, 210, 230, 250, 280, and 300.

### EXAMPLE EMBODIMENT

Practice of the invention will be still more fully understood from the following example, which is presented herein for illustration only and should not be construed as limiting the invention in any way.

A carrier device for delivery of drugs to a diarthrodial joint of a human or animal subject. The carrier device includes multiple structural members. The multiple structural members are adapted to: secure the carrier device to the tissues of the joint space to stabilize the device within the joint, and for delivering a payload of a separate device. In an approach, the multiple structural members comprise: one or more longitudinal core members; and one or more endcap members. The one or more endcap members may have suture eyelets, and the endcap members may be affixed to the longitudinal core members. The suture eyelet members maybe affixed to the endcap members, and the longitudinal core members and endcap members are adapted to delineate a payload space for the payload device. The carrier device may be secured inside the diathrodial joint of a human or animal, and the multistructural structures may comprise a suture for the securing the device. The suture attaches to the carrier device by the suture eyelets. The geometry of the carrier device allows it to be inserted through small incisions of the human or animal subject. The securing of the carrier device with the suture comprises attaching the suture to bony and soft tissue structures within the joint of the human or animal subject to secure the device. The attaching of the sutures comprises at least one of the following methods: suturing joint capsule, open knot tying, arthroscopic knot tying, or bone anchors. At least part of the carrier device comprises at least one of or any combination of the following materials: metals, ceramics, or polymers. At least part of the carrier device comprises at least on of or any combination of the following materials: bioerodible and/or biodegradable polymers, co-polymers, blends of polymers or composite devices which are biocompatible. The longitudinal core members is longer than wide; wherein the endcap members have width equal to or less than the length of the longitudinal core members. Further, the carrier device is adapted to be inserted longitudinally through the incision that is as long or longer than the width of the endcap members. The longitudinal core members comprise geometries including at least one of the following geometries: cylinder, prism, cylindrical shell, section of a pyramidal solid, or partial arc of these geometries. The longitudinal core members may be circumferentially integrated by means of hoops or a screen or mesh for protection of payload. The cap members comprise geometries including at least one of the following geometries: concave-concave disc, concave-convex disc, convex-convex disc, cylinder, a prism, rotated base, extruded base, or a fillet-modified version of the above geometries.

### REFERENCES CITED

It should be appreciated that various aspects of embodiments of the present method and device/system may be implemented with the following methods and devices/systems disclosed in the following U.S. Patent Applications, U.S. Patents, and PCT International Patent Applications that are hereby incorporated by reference herein.

### U.S. Patent Documents

1. U.S. Pat. No. 6,530,934 B1, Jacobsen, et. al., “Embolic Device Composed of Linear Sequence of Miniature Beads”, Mar. 11, 2003.

2. U.S. Pat. No. 5,466,233, Weiner, et. al., “Tack for Intraocular Drug Delivery and Method for Inserting and Removing Same”, Nov. 14, 1995.

3. U.S. Pat. No. 6,901,287, Davis, et. al., “Implantable Therapy Delivery Element Adjustable Anchor”, May 31, 2005.

4. U.S. Pat. No. 5,281,419, Tuan, et. al., “Biodegradable Drug Delivery System for the Prevention and Treatment of Osteomyelitis”, Jan. 25, 1994.

5. U.S. Pat. No. 5,756,127, Grisoni, et. al., “Implantable Bioresorbable String of Calcium Sulfate Beads”, May 26, 1988.

6. U.S. Pat. No. 5,919,473, Elkhoury, “Methods and Devices for Delivering Opiod Analgesics to Wounds Via A Subdermal Implant”, Jul. 6, 1999.

7. U.S. Pat. No. 6,913,760 B2, Carr, et. al., “Drug Delivery Composition”, Jul. 5, 2005.

8. U.S. Pat. No. 5,922,340, Berde, et. al., “High Load Formulations and Methods for Providing Prolonged Local Anesthesia”, Jul. 13, 1999.

9. U.S. Pat. No. 6,921,541 B2, Chasin, et. al., “Formulations and Methods for Providing Prolonged Local Anesthesia”, Jul. 26, 2005.

10. U.S. Pat. No. 6,699,908 B2, “Sackler, et. al., “Methods for Providing Safe Local Anesthesia”, Mar. 2, 2004.

11. U.S. Pat. No. 7,052,719 B2, Bernstein, et. al., “Matrices Formed of Polymer and Hydrophobic Compounds for use in Drug Delivery”, May 30, 2006.

12. U.S. Pat. No. 6,534,081 B2, Goldenheim, et. al., “Prolonged Anesthesia in Joints and Body Spaces”, Mar. 18, 2003.

13. U.S. Pat. No. 7,037,324 B2, Martinek, et. al., “Knotless Tissue Anchor”, May 2, 2006.

14. U.S. Pat. No. 7,001,411 B1, Dean, “Soft Tissue Cleat”, Feb. 21, 2006.

15. U.S. Pat. No. 6,932,834 B2, Lizardi, et. al., “Suture Anchor”, Aug. 23, 2006.

16. U.S. Pat. No. 6,923,823 B1, Bartlett, et. al., “Apparatus and Method for Anchoring Sutures”, Aug. 2, 2005.

17. U.S. Pat. No. 6,749,620 B2, Bartlett, “Apparatus and Method for Anchoring Sutures”, Jun. 15, 2004.

18. U.S. Pat. No. 6,592,609 B1, Bonutti, “Method and Apparatus for Securing Tissue”, Jul. 15, 2003.

19. U.S. Pat. No. 6,626,916 B1, Yeung, et. al., “Tissue Fastening Devices and Methods for Sustained Holding Strength”, Sep. 20, 2003.

### U.S. Patent Application Publication Documents

1. U.S. Patent Application Publication No. 2005/0031665 A1, Watson, et. al., “Device and Method for Treating Conditions of a Joint”, Feb. 10, 2005.

2. U.S. Patent Application Publication No. 2002/0169162 A1, Smith, et. al., “Device and Method for Treating Conditions of a Joint”, Nov. 14, 2002.

3. U.S. Patent Application Publication No. 2005/0152949 A1, Hotchkiss, et. al., “Drug Delivery to a Joint”, Jul. 14, 2005.

4. U.S. Patent Application Publication No. 2005/0158365 A1, Watson, et. al., “Drug Delivery Device with Mechanical Locking Mechanism”, “Jul. 21, 2005.

5. U.S. Patent Application Publication No. 2005/0136095 A1, “Levy, et. al., “Drug Delivery Device with Suture Ring”, Jun. 23, 2005.

6. U.S. Patent Application Publication No. 2005/0113806 A1, “De Carvalho, et. al., “Implantable and Sealable Medical Device for Unidirectional Delivery of Therapeutic Agents to Tissues”, May 26, 2005.

7. U.S. Patent Application Publication No. 2004/0009228 A1, Tormala, et. al., “Bioabsorbable Drug Delivery System for Local Treatment and Prevention of Infections”, Jan. 15, 2004.

8. U.S. Patent Application Publication No. 2005/0267565 A1, Dave, et. al., “Biodegradable Medical Implant with Encapsulated Buffering Agent”, Dec. 1, 2005.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents. Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A carrier device for delivery of drugs to a diarthrodial joint of a human or animal subject, said carrier device comprising:
multiple structural members, said multiple structural members adapted to:
secure said carrier device to the tissues of the joint space to stabilize said device within the joint, and for delivering a payload of a separate device.

2. The carrier device of claim 1, wherein said multiple structural members comprise:
one or more longitudinal core members; and
one or more endcap members, said one or more endcap members having suture eyelets, and wherein: said endcap members are affixed to said longitudinal core members, and the suture eyelet members are affixed to the endcap members, and said longitudinal core members and endcap members are adapted to delineate a payload space for the payload device.

3. The carrier device of claim 2, wherein said carrier device being secured inside the diathrodial joint of a human or animal, and said multistructural structures comprising a suture for said securing the device.

4. The carrier device of claim 3, wherein said aid suture attaches to said carrier device by said suture eyelets.

5. The carrier device of claim 2, wherein at least part of said carrier device comprises at least one of or any combination of the following materials: metals, ceramics, or polymers.

6. The carrier device of claim 2, wherein at least part of said carrier device comprises at least on of or any combination of the following materials: bioerodible and/or biodegradable polymers, co-polymers, blends of polymers or composite devices which are biocompatible.

7. The carrier device of claim 3, wherein the geometry of said carrier device allows it to be inserted through small incisions of the human or animal subject.

8. The carrier device of claim 3, wherein said longitudinal core members is longer than wide; wherein said endcap members have width equal to or less than the length of the longitudinal core members; and whereby said carrier device is adapted to be inserted longitudinally through the incision that is as long or longer than the width of said endcap members.

9. The carrier device of claim 2, wherein said longitudinal core members comprise geometries including at least one of the following geometries: cylinder, prism, cylindrical shell, section of a pyramidal solid, or partial arc of these geometries.

10. The carrier device of claim 2, wherein said longitudinal core members may be circumferentially integrated by means of hoops or a screen or mesh for protection of payload.

11. The carrier device of claim 2, wherein said end cap members comprise geometries including at least one of the following geometries: concave-concave disc, concave-convex disc, convex-convex disc, cylinder, a prism, rotated base, extruded base, or a fillet-modified version of the above geometries.

12. The carrier device of claim 3, wherein securing said carrier device with said suture comprises: attaching said suture to bony and soft tissue structures within the joint of the human or animal subject to secure said device.

13. The carrier device of claim 12, wherein said attaching said sutures comprises at least one of the following methods: suturing joint capsule, open knot tying, arthroscopic knot tying, or bone anchors.

14. A method for delivery of drugs to a diarthrodial joint of a human or animal subject using a carrier device, said method comprising:
securing said carrier device to the tissues of the joint space to stabilize said device within the joint, and
delivering a payload of a separate device.

15. The method of claim 14, wherein said carrier device adapted for providing a payload space for accommodating the payload device.

16. The method of claim 14, wherein said securing said carrier device to the tissues comprises securing said carrier device inside the diathrodial joint of a human or animal.

17. The method of claim 16, wherein said securing of said carrier device inside the diathrodial joint of a human comprises at least one suture.
